(19) United States
(12) Patent Application Publication
(10) Pub. No.: US 2025/0099266 A1
(43) Pub. Date: Mar. 27, 2025

(54) PROSTHETIC IMPLANT OF THE ESR TYPE AND METHOD FOR ADJUSTING THE PROSTHETIC IMPLANT

(71) Applicant: ALMA MATER STUDIORUM - UNIVERSITA' DI BOLOGNA, BOLOGNA (IT)

(72) Inventors: JOHNNIDEL TABUCOL, MINERBIO (IT); MARCO LEOPALDI, MARANO DI NAPOLI (IT); TOMMASO MARIA BRUGO, BOLOGNA (IT); ANDREA ZUCCHELLI, ZOLA PREDOSA (IT)

(21) Appl. No.: 18/728,178

(22) PCT Filed: Jan. 10, 2023
(86) PCT No.: PCT/IB2023/050195

§ 371 Date: Jul. 11, 2024

(30) Foreign Application Priority Data
Jan. 13, 2022 (IT) 102022000000485

(51) Int. Cl. A61F 2/66; A61F 2/70

(52) U.S. Cl. CPC A61F 2/6607; A61F 2002/6614; A61F 2002/701

(57) ABSTRACT

A prosthetic implant of the ESR (energy-storing-and-returning) type, includes an upper assembly and a lower assembly, configured to define a foot and including a lower blade, an intermediate blade and an upper blade. The upper blade, the lower blade and the intermediate blade are fastened stably to each other at at least one connecting point located in the front region or in the middle region of the foot. An articulated joint is interposed between the lower assembly and the upper one to allow relative motion between them. The implant includes a connector having a first end that is connected to the upper assembly and a second end that is connected to the intermediate blade of the lower assembly. At least one between the first and the second end of the connector is movable along a movement direction to vary the force applied on the intermediate blade of the lower assembly.

### TECHNICAL FIELD

This invention relates to a prosthetic implant of the ESR (energy-storing-and-returning) type and to a method for adjusting a prosthetic implant of the ESR type which is widely used in the medical field.

In particular, this invention is widely used in the field of lower limb prostheses and, more specifically, in the field of foot prostheses.

### BACKGROUND ART

As is known, during walking, the foot undergoes a load cycle, depending mainly (but not only) on the conditions of the ground, the person's weight, a load being carried, if any, and the walking speed, to which the foot reacts by adapting its rigidity.

To date, therefore, there is a particularly strongly felt need to ensure that amputees have their ambulating functions restored almost entirely to the conditions existing prior to amputation. To meet this need, various different prosthetic implants are available as substitutes which aim to replicate as faithfully as possible the complexities of the force-deformation curve representing a patient's natural gait cycle.

In other words, the aim, to date, is to create a prosthetic implant that is capable of providing a mechanical reaction to different ground types, different obstacles and, generically, different loads, that is as close as possible to the mechanical reaction of a natural foot.

In this situation, various different prosthetic implants have been developed which, by varying the stiffness of the implant itself, allow the patient to walk not only on flat ground, but also on irregular surfaces such as, for example, footpaths, uneven ground, stairs, ramps and the like.

An example of such prosthetic implants is disclosed in U.S. Ser. No. 10/034,782B2. That patent document discloses an implant comprising an elongate prosthetic foot element in the form of a blade provided with a longitudinal slot. The implant also comprises a tongue portion configured to flex independently of the foot element. The prosthetic implant also includes a selectively actuated mechanism to operatively connect/disconnect the tongue portion to/from the foot element. More in detail, when the tongue portion is operatively connected to it, the foot element exhibits greater stiffness, and when the tongue portion is operatively disconnected from it, the foot element exhibits lower stiffness.

In other words, coupling and uncoupling the tongue portion and the foot element to and from each other allows varying the stiffness of the implant so as to adapt it to the patient's ambulation requirements.

Disadvantageously, actuating the mechanism which connects/disconnects the tongue portion is particularly complex and difficult, especially if the mechanism needs to be actuated when the prosthetic implant is in use.

Another disadvantage is due to the fact that in the prosthetic implant described in U.S. Ser. No. 10/034,782B2, the variable stiffness is limited to the foot element only. That means the implant is less flexible and less adaptable to the patient's ambulation requirements in that it does not allow varying and adjusting the torsional stiffness of the ankle portion of the implant.

This drawback is at least partly overcome by the prosthetic implant disclosed in U.S. Pat. No. 9,289,316B2. That patent document discloses an implant comprising a main body (or ankle) coupled to a foot by an articulated system. The articulated system acts as a variable lever arm for adapting the response of the implant to the patient's movement.

In particular, the articulated system comprises a spring having one end hinged to the main body at a fixed hinge point and another end engaged with the foot by a mounting block that is slidable along a horizontal direction (parallel to the patient's direction of movement).

When the patient walks, the sliding of the mounting block causes a variation of the length of the lever arm defined by the articulated system. Adjusting the lever arm makes it possible to control the torsional stiffness of the articulated system. More specifically, the torsional stiffness is linked to the linear stiffness of the spring and to the length of the lever arm. Thus, changing the position of the mounting block has the effect of changing the torsional stiffness of the prosthetic implant.

Disadvantageously, since the torsional stiffness of the prosthetic implant depends on a spring with linear stiffness, the prosthetic implant of U.S. Pat. No. 9,289,316B2 is unable to precisely and reliably mimic the non-linear trend of the force-deformation curve representing the patient's natural gait cycle. This drawback is at least partly overcome by prosthetic implants whose torsional stiffness can be varied by a system of shock absorbers operating with thickening fluids which, by varying their viscosity, allow the prosthetic implant to respond adaptively.

In other words, depending on the phase of the load cycle the prosthetic implant is subjected to (that is, depending on which phase of their gait cycle the patient is in), the viscosity of the thickening fluids varies to allow the implant to react in an optimal manner.

Disadvantageously, these prosthetic implants also have drawbacks. In particular, as the patient walks, for the prosthetic implant to respond adequately to load variations due, for example, to stairs, uneven ground, obstacles and the like, the response reaction of the implant with the shock absorber system needs to be well calibrated. That means that the energy that might be accumulated in an unloading phase of the cycle (known as “stance phase”) is lost and thus cannot be used by the patient in the step that follows (in what is known as the “push-off phase”). In this situation, prosthetic implants that exploit the viscosity variation of thickening fluids are uncomfortable to use and have a considerable fatiguing effect on the patient.

Other examples of foot prostheses are known from patent documents EP3128958A1 and US2020/375764A1. EP3128958A1 involves varying the length of the element that connects the top plate assembly to the blades. Even these solutions, however, do not allow the stiffness to be adjusted in a simple and precise manner.

Also known are fully actuated prostheses which can vary the stiffness but which require the actuating motor to work at each step the patient takes. This has an enormous impact on mobility.

The technical purpose of this invention, therefore, is to provide a prosthetic implant of the ESR (energy-storing-and-returning) type and a method for adjusting a prosthetic implant of the ESR type to overcome the above mentioned disadvantages of the prior art.

### DISCLOSURE OF THE INVENTION

The aim of this invention, therefore, is to provide a prosthetic implant of the ESR (energy-storing-and-returning) type, but also of the semi-active ESR and active ESR type, capable of replicating as faithfully as possible the force-deformation curve representing the patient's natural gait cycle.

Another aim of this invention is, therefore, to provide a prosthetic implant of the ESR (energy-storing-and-returning) type that is comfortable for the user and reliable in use.

The technical purpose indicated and the aims specified are substantially achieved by a prosthetic implant of the ESR (energy-storing-and-returning) type and a method for adjusting a prosthetic implant of the ESR type, comprising the technical features set out in one or more of the appended claims. The dependent claims correspond to possible embodiments of the invention.

More specifically, the technical purpose claimed and the aims specified are achieved by a prosthetic implant of the ESR (energy-storing-and-returning) type. The prosthetic implant comprises an upper assembly configured to be connected to a patient's lower limb, and a lower assembly configured to define a foot.

The foot has a front region, a middle region and a rear region. The lower assembly includes a lower blade, extending along a main direction between a heel end and a toe end.

According to an aspect of the disclosure, the lower blade, along the main direction, has a curved shape that replicates the curvature of a sole of a foot.

According to another aspect of the disclosure, the lower blade is provided, along the main direction, with a longitudinal slot extending from the toe end substantially up to the middle region of the foot.

The slot divides the toe end into two rounded portions defining a “forefoot portion” (that is, the part defining the toes of the foot).

The lower assembly also comprises an intermediate blade that is partly superposed on the lower blade, preferably in the middle region of the foot. In the preferred embodiment, the intermediate blade has a first straight stretch extending from a toe end of the intermediate blade and at least partly superposed on the lower blade.

In the preferred embodiment, the intermediate blade also has a second straight stretch extending at an angle to the first stretch. The second stretch extends from the first stretch towards a heel end of the intermediate blade. According to an aspect of the disclosure, the intermediate blade also has a longitudinal slot, extending from the heel end to a middle zone of the intermediate blade itself.

The lower assembly also comprises an upper blade that is partly superposed on the intermediate blade, preferably in the middle region of the foot.

In the preferred embodiment, the upper blade is substantially rectilinear in shape.

The upper blade, the intermediate blade and the lower blade are superposed on each other substantially in the middle region of the foot. More precisely, the upper blade, the intermediate blade and the lower blade are superposed on each other between the middle region and the front region of the foot.

The upper blade, the intermediate blade and the lower blade are fastened stably to each other at at least one connecting point located in the front region or in the middle region of the foot.

In a possible embodiment, the connecting point comprises two parallel pins extending from the upper blade to the lower blade through the intermediate blade.

The upper, lower and intermediate blades diverge from each other away from the connecting point towards the rear region of the foot.

The upper, lower and intermediate blades are laminated springs made from composite material. In this situation, the upper, lower and intermediate blades have a non-linear elastic behaviour.

The prosthetic implant also comprises an articulated joint, interposed between the lower assembly and the upper one to allow relative motion between the lower assembly and the upper one.

The articulated joint acts as a ball joint allowing the patient to walk, that is to say, to mimic an ankle joint.

In a possible embodiment, the articulated joint includes a ball that is stably fixed to the upper assembly and faces towards the lower assembly. The articulated joint also includes a receiving body that is fixed to the lower assembly and faces towards the upper assembly to receive the ball. The receiving body is fixed to the upper blade in the proximity of its heel end.

In a possible embodiment, the receiving body is distinct from the lower assembly, and more specifically, from the upper blade, and is fastened to the lower assembly by, for example, screws or the like.

Alternatively, the receiving body is made as a single part with the upper blade.

The ball and the receiving body define a ball and socket joint.

Alternatively, the ball and the receiving body define a universal joint or a simple hinge.

In an example, the ball and the receiving body are made from composite material. Alternatively, the ball and the receiving body may be made from a material customarily used to make prostheses.

The prosthetic implant also comprises a connector having a first end that is connected to the upper assembly, and a second end that is connected to the intermediate blade of the lower assembly to apply a force on the intermediate blade of the lower assembly.

The connector acts substantially as a tendon element configured for transmitting a force between the upper assembly and the lower assembly. In a possible embodiment, the connector comprises a first portion which extends from the first end and a second portion which extends from the second end and which is configured to be connected to the first portion.

The first and the second portion define a lead nut and screw connection.

According to an aspect of this disclosure, the connector is a rigid element of adjustable length.

According to a possible example embodiment, the length of the connector is adjustable manually. According to another embodiment, the length might be adjustable automatically by means of a motor.

According to another aspect of this disclosure, at least one between the first and the second end of the connector is movable along a movement direction to vary the force applied on the intermediate blade of the lower assembly. In this situation, it is possible to vary the stiffness of the foot by adapting it easily and precisely to the different loads the patient is subjected to and/or to the different types of ground the patient walks on. The connector is elongated along a connection axis. The movement direction is inclined with respect to the connection axis. The movement direction is also inclined with respect to the longitudinal direction. Preferably, the movement direction is perpendicular to the longitudinal direction. The inclination angle between the movement direction and the connection axis varies with the changing position of the movable end of the connector; for example, the angle might be ninety degrees (or slightly greater); more generally speaking and by way of example, the angle might vary between 70 (or 90) and 130 degrees. It should be noted that the change in position (displacement) of the at least one between the first and the second end of the connector along a movement direction does not, in itself, involve any change of length of the connector (the connector might even be of fixed length). Preferably, it is the first end of the connector that is movable along the movement direction (that is, along a movement axis).

More in detail, being able to control the change in the connector fastening position where the force is exchanged between the lower assembly (comprising the blades, which have non-linear behaviour) and the upper assembly makes it possible to vary the point where the force is applied. In this situation, the lever arm of the force relative to the centre of rotation of the ankle changes, hence the transmitted torque also changes. By so doing, the variation in the torsional stiffness obtained can be attributed to the entire foot and is a non-linear variation, that is, a variation that replicates the natural behaviour of a foot in a highly faithful manner.

In an embodiment, the upper assembly extends along a longitudinal direction between a hook-up end, where it is operatively connected to the patient's lower limb, and a fixing end, where it is fixed to the articulated joint. The first end of the connector is slidably movable relative to the upper assembly and the movement direction is transverse to the longitudinal axis. More specifically, the movement direction is perpendicular to the longitudinal axis.

In the preferred embodiment, when the first end of the connector is slidably movable relative to the upper assembly, the second end of the connector is articulated to the intermediate blade of the lower assembly.

More specifically, the second end of the connector is hinged to the intermediate blade of the lower assembly so that a sliding movement of the first end of the connector corresponds to a rotational movement of the connector about the second end.

According to an aspect of the disclosure, the prosthetic implant comprises a slider that is extractable or retractable relative to the upper assembly along the movement direction through a plurality of equilibrium positions.

The first end of the connector is pivoted to the slider to adjust the position of the first end itself between a most extracted position, where the first end is distal to the upper assembly, and a least extracted position, where the first end is proximal to the upper assembly.

At the most extracted position, the slider is extracted and the connector is rotated about the second end so that the first end is distal to the upper assembly. In this situation, the prosthetic implant exhibits a high value of stiffness.

At the least extracted position, the slider is retracted and the connector is rotated about the second end so that the first end is close to the upper assembly. In this situation, the prosthetic implant exhibits a lower value of stiffness.

The slider might also be partly extracted from the upper assembly to occupy an intermediate position between the most extracted position and the least extracted position. The intermediate position corresponds to a walking state of the patient.

Instead of the slider, the prosthetic implant might, for example, comprise a cam capable of varying the position of the first end of the connector as described above. In use, therefore, by controlling (by extracting or retracting the slider) the position of the fastening point (that is, of the first end) of the connector, where the force is exchanged between the lower assembly and the upper assembly, it is possible to vary the point where the force is applied. This also changes the lever arm of the force relative to the centre of rotation of the ankle (articulated joint), thus varying the transmitted torque, hence the torsional stiffness of the entire foot. Since the blades have a non-linear elastic behaviour, the variation in the stiffness obtained is non-linear and such as to better replicate the force-deformation curve of a natural foot subjected to different states of stress and/or loads (for example, uneven ground, weights lifted, and so on).

In the preferred embodiment, the slider is coupled to the upper assembly by a prismatic coupling.

Alternatively, the slider may be coupled to the upper assembly by any other coupling that allows translational motion in the desired direction and is constrained in the other translational and rotational movements.

In a possible embodiment, the first end is movable slidably via an actuator which is, for example, connected to the upper assembly.

Alternatively, the first end is movable slidably by manual operation.

In another possible embodiment, the first end of the connector may be articulated to the upper assembly—hinged to it, for example—while the second end may be movable slidably along the intermediate blade.

In this situation, the prosthetic implant comprises a slide that is slidably movable along the intermediate blade to steplessly vary the position of the second end of the connector on the intermediate blade.

The prosthetic implant also comprises an adjustment element that is activable on the slide to adjust the position of the slide on the intermediate blade. In this situation, varying the position of the second end of the connector makes it possible to vary the point where the force is applied. This also changes the lever arm of the force relative to the centre of rotation of the ankle (articulated joint), thus varying the transmitted torque, hence the torsional stiffness of the entire foot.

The technical purpose and the aims specified are also achieved by a method for adjusting a prosthetic implant of the ESR (energy-storing-and-returning) type. The prosthetic implant comprises an upper assembly configured to be connected to a patient's lower limb, and a lower assembly configured to define a foot having a front region, a middle region and a rear region. The lower assembly includes a lower blade, extending along a main direction between a heel end and a toe end.

The lower assembly includes an intermediate blade that is partly superposed on the lower blade, preferably in the middle region of the foot. The lower assembly includes an upper blade that is partly superposed on the intermediate blade, preferably in the middle region of the foot.

The blades are laminated springs made from composite material.

The upper, lower and intermediate blades are fastened stably to each other at at least one connecting point located in the front region or in the middle region of the foot and diverge from each other away from the connecting point towards the rear region of the foot.

The prosthetic implant also comprises an articulated joint—for example, a ball and socket joint, a rotoidal joint or a universal joint—interposed between the lower assembly and the upper assembly to allow relative motion between the lower assembly and the upper assembly.

The prosthetic implant also comprises a connector having a first end that is connected to the upper assembly, and a second end that is connected to the intermediate blade of the lower assembly to apply a force on the intermediate blade of the lower assembly.

More in detail, the connector acts as a rigid tendon element capable of transmitting a force between the upper assembly and the lower assembly. The method comprises a step of moving at least one between the first and the second end of the connector to vary the force applied by the connector on the intermediate blade of the lower assembly.

In the preferred embodiment, in the step of moving, it is the first end that moves, while the second end is articulated to the intermediate blade—hinged to it, for example.

In an embodiment, the upper assembly extends along a longitudinal direction between a hook-up end, where it is operatively connected to the patient's lower limb, and a fixing end, where it is fixed to the articulated joint. In this situation, the method comprises a step of sliding the first end of the connector relative to the upper assembly. In this situation, the movement direction is transverse, specifically perpendicular, to the longitudinal axis.

In other words, to vary the force applied by the connector on the intermediate blade of the lower assembly, the first end of the connector is moved slidably along the movement direction.

The prosthetic implant also comprises a slider, to which the first end of the connector is pivoted. In this situation, the method also comprises a step of extracting or retracting the slider relative to the upper assembly along the movement direction through a plurality of equilibrium positions. The method also comprises a step of adjusting the position of the first end of the connector between a most extracted position, where the first end is distal to the upper assembly, and a least extracted position, where the first end is proximal to the upper assembly.

In other words, by extracting or retracting the slider relative to the upper assembly, it is possible to change in a controlled manner the position of the first end of the slider, where the force is exchanged between the foot and the upper assembly. Changing the position of the first end of the slider, that is, the point where the force is applied, changes the lever arm of the force relative to the centre of rotation of the ankle, hence the transmitted torque. In this situation, the blades change their point of rotation with the changing of the position of the first end of the connector, thus obtaining a non-linear variation of the torsional stiffness of the entire foot, capable of faithfully replicating the behaviour of a foot when subjected to stresses due to different loads and/or different ground conditions.

Further features and advantages of this invention are more apparent in the exemplary, hence non-limiting, description of an embodiment of a prosthetic implant of the ESR (energy-storing-and-returning) type and a method for adjusting a prosthetic implant of the ESR type.

### BRIEF DESCRIPTION OF THE DRAWINGS

The description is set out below with reference to the accompanying drawings which are provided solely for purposes of illustration without limiting the scope of the invention and in which:

FIG. 1 shows a perspective view of the prosthetic implant;

FIG. 1A shows a detail from FIG. 1;

FIGS. 2A-2C show a side view of the prosthetic implant in three different configurations;

FIG. 3 shows a perspective view of the prosthetic implant of FIG. 1, with an actuator applied to it;

FIG. 4 is a side view of a further embodiment of a prosthetic implant;

FIG. 4A shows a detail from FIG. 4.

### DETAILED DESCRIPTION OF PREFERRED EMBODIMENTS OF THE INVENTION

With reference to the accompanying drawings, the numeral 100 denotes a prosthetic implant of the ESR (energy storing and returning) type.

The prosthetic implant 100 comprises an upper assembly 200 configured to be connected to a patient's lower limb.

The upper assembly 200 extends along a longitudinal direction A between a hook-up end 200a, where it is operatively connected to the patient's lower limb, and a fixing end 200b, where it is fixed to an articulated joint 500.

The prosthetic implant 100 also comprises a lower assembly 300, configured to define a foot having a front region 300a, a middle region 300b and a rear region 300c.

The lower assembly 300 includes a lower blade 400a, extending along a main direction M, between a heel end 400a′ and a toe end 400a″.

In use, the main direction M coincides with the direction of forward movement of the patient while walking.

According to an aspect of this disclosure, the lower blade 400a has a curved shape along the main direction M, where the heel end 400a′ and the toe end 400a″ rest on the ground, while the part of the lower blade 400a between them is at least partly raised off the ground.

In other words, the lower blade 400a simulates the curvature and shape of the sole of a foot.

According to another aspect of this disclosure, the lower blade 400a has a slot I extending longitudinally on the lower blade 400a. In this situation, in the proximity of the toe end 400a″, the slot I divides the lower blade 400a into two rounded portions L.

The lower assembly 300 also comprises an intermediate blade 400b, partly superposed on the lower blade 400a.

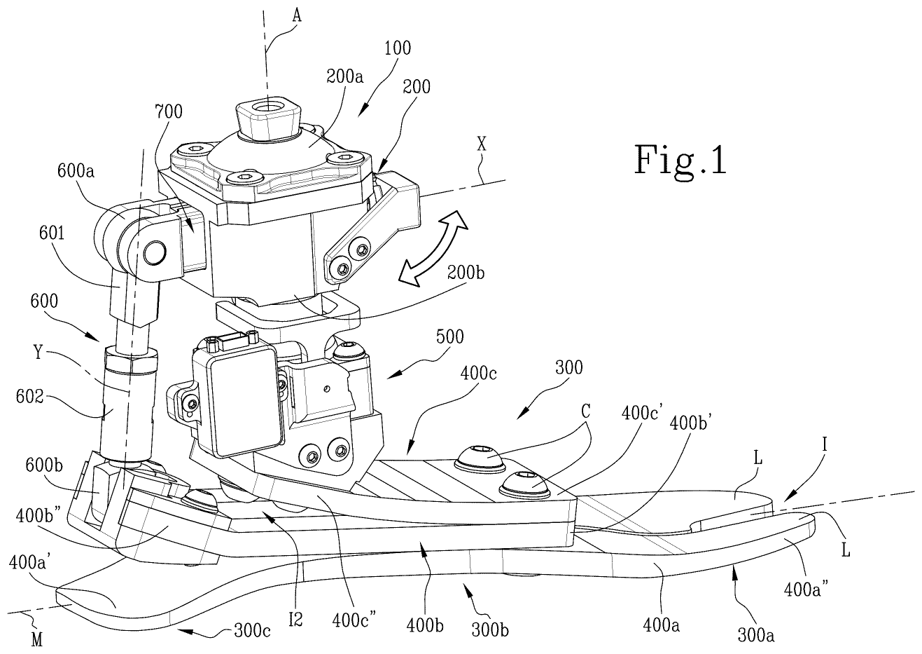

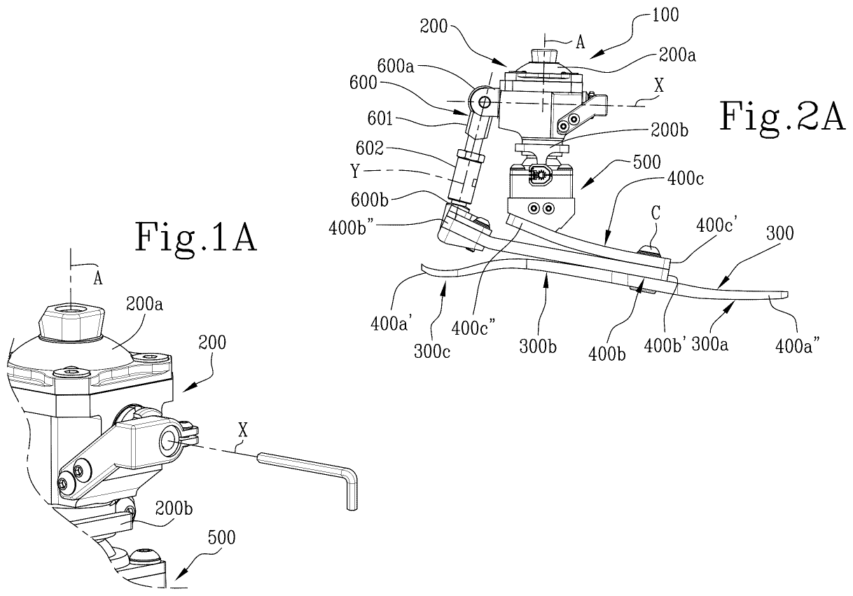

More specifically, as shown, for example, in FIG. 1, the intermediate blade 400b is superposed on the lower blade 400a in proximity to the middle region 300b of the foot.

The intermediate blade 400b has a first, substantially rectilinear stretch extending from a toe end 400b′ and a second stretch, lying at an angle to the first stretch and extending between the first stretch and a heel end 400b″.

The intermediate blade 400b too is provided with a slot 12 extending longitudinally between the middle region 300b of the foot and the heel end 400b″ of the intermediate blade 400b.

The lower assembly 300 also comprises an upper blade 400c, partly superposed on the intermediate blade 400b.

More specifically, as shown, for example, in FIG. 1, the upper blade 400c is superposed on the intermediate blade 400b in proximity to the middle region 300b of the foot.

The upper blade 400c has a slight curvature extending from a toe end 400c′ to a heel end 400c″.

The upper blade 400c is shorter in length than the intermediate blade 400b. The intermediate blade 400b is shorter in length than the lower blade 400a. The upper, lower and intermediate blades 400a, 400b, 400c are fastened stably to each other at at least one connecting point C located in the front region 300a or in the middle region 300b of the foot and diverge from each other away from the connecting point C towards the rear region 300c of the foot.

In the embodiment shown in the accompanying drawings, the connecting point C is located in the middle region 300b of the foot.

In a possible embodiment, the connecting point C is embodied by two parallel pins extending between the upper blade 400c and the lower blade 400a.

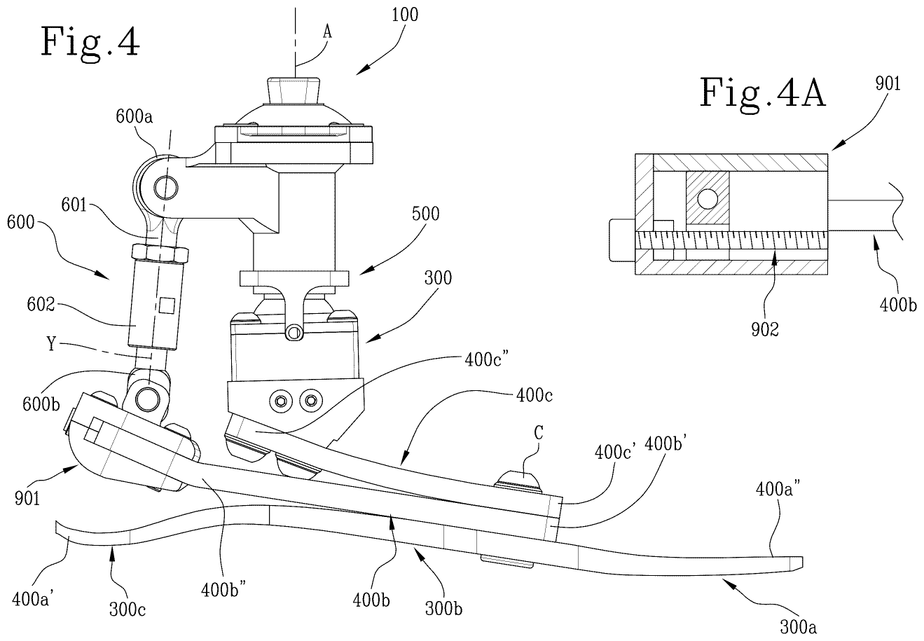

Aa shown, for example, in FIGS. 1 and 4, the upper blade 400c and the intermediate blade 400b are connected at the connecting point C in proximity to their respective toe ends 400b′, 400c′.

The lower blade 400a, on the other hand, is connected to the other blades 400b, 400c in proximity to the middle region 300b of the foot.

According to an aspect of this disclosure, the lower blade 400c, the intermediate blade 400b and the upper blade 400a are laminated springs made from composite material.

When subjected to stress, the lower blade 400a, the intermediate blade 400b and the upper blade 400c have a non-linear elastic behaviour.

The prosthetic implant 100 also comprises an articulated joint 500, interposed between the lower assembly 300 and the upper one 200 to allow relative motion between the lower assembly 300 and the upper one 200.

In a possible embodiment, the articulated joint 500 includes a ball that is stably fixed to the upper assembly 200 and faces towards the lower assembly 300.

The articulated joint 500 also includes a receiving body that is fixed to the lower assembly 300 and faces towards the upper assembly 200 to receive the ball. In this situation, the ball and the receiving body define a ball and socket joint.

In the embodiment shown in the accompanying drawings, the receiving body is stably fixed to the upper blade 400c, preferably in the rear region 300c of the foot and in proximity to the heel end 400c″ of the upper blade 400c itself.

The ball and socket joint 500 allows the upper assembly 200 and the lower assembly 300 to move relative to each other in that the ball can roll in the receiving body.

In other words, the ball and socket joint 500 simulates the patient's ankle articulation.

According to an aspect of this disclosure, the prosthetic implant 100 also comprises a connector 600 having a first end 600a that is connected to the upper assembly 200 and a second end 600b that is connected to the intermediate blade 400b of the lower assembly 300 to apply a force on the intermediate blade 400b of the lower assembly 300, as described in detail below. The connector 600 extends from the first end 600a to the second end 600b along a connection axis Y.

The connector 600 is located in proximity to the rear region 300c of the foot.

According to an aspect of this disclosure, one between the first and the second end 600a, 600b of the connector 600 is movable along a movement direction X to vary the force applied on the intermediate blade 400b of the lower assembly 300.

The movement direction X is inclined with respect to the connection axis Y. The movement direction X is inclined (preferably perpendicular) with respect to the longitudinal direction A. Thus, the displacement of the at least one between the first and the second end along the movement direction X causes the first or the second end of the connector 600 to move towards or away from the longitudinal axis A.

The connector 600 constrains the upper assembly 200 and the lower assembly 300 to rotate in a plane defined by the longitudinal axis A and by the movement direction, as shown by the arrow in FIG. 1.

In other words, the connector 600 causes the articulated joint 500 to act as a centre of rotation for the prosthetic implant 100.

According to an aspect of this disclosure, the connector 600 is a rigid element.

According to another aspect of this disclosure, the connector 600 is adjustable (for example, it is variable in length).

Looking in more detail, the connector 600 comprises a first portion 601 extending away from the first end 600a, and a second portion 602 extending away from the second end 600b and engaged with the first portion 601.

The first and the second portion 601, 602 are engaged with each other by a lead nut and screw coupling configured to set a length for using the connector 600 and to connect the first and the second portion 601, 602 rigidly so that the connector 600 acts as a rigid arm of preset length.

In this situation, by changing the position of one between the first and the second end 600a, 600b of the connector 600 it is possible to obtain, through the connector 600 itself, a variable lever arm so as to adapt the response of the prosthetic implant 100 as the patient moves.

In effect, by varying the lever arm, it is possible to change the point of rotation (or of flexion) of the blades 400a, 400b, 400c, varying their state of stress. Since the blades 400a, 400b, 400c are elastically deformable in non-linear manner, varying the lever arm makes it possible to vary the stiffness of the prosthetic implant 100 in non-linear manner so that the behaviour of the prosthetic implant 100 under stress is as close as possible to that of a natural foot.

In other words, changing the position of the point where the force is applied, changes the lever arm of the force relative to the centre of rotation of the articulated joint 500, hence the transmitted torque. The variation in the torsional stiffness obtained can be attributed to the entire foot, that is to the entire lower assembly 300.

In a possible embodiment shown in FIGS. 1, 2A-2C, 3, the first end 600a of the connector 600 is slidably movable relative to the upper assembly 200. In this situation, the movement direction X is transverse to the longitudinal axis A.

In the preferred embodiment, the movement direction X is perpendicular to the longitudinal direction A.

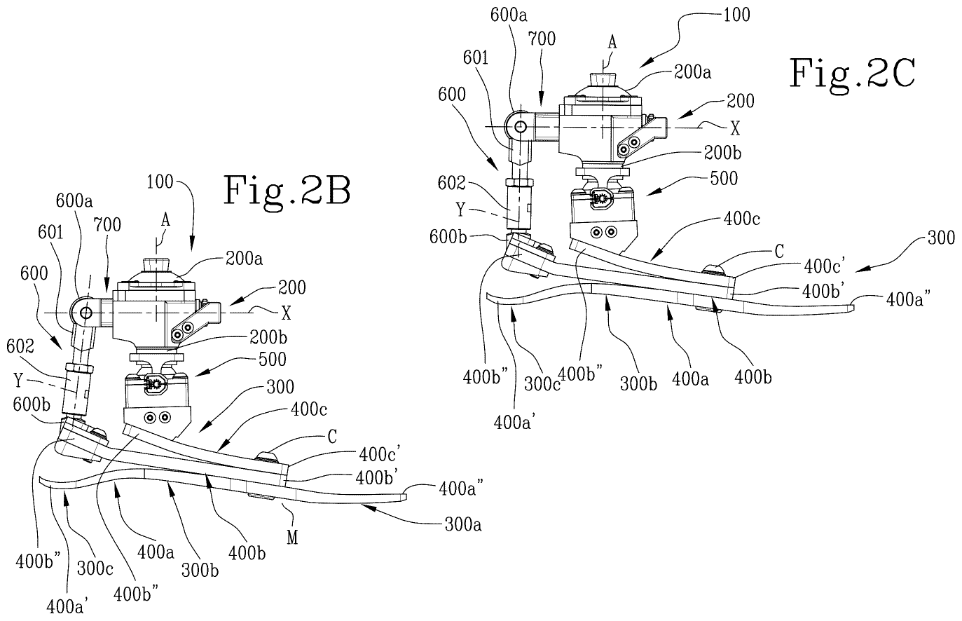

As shown in FIGS. 2A-2C, the prosthetic implant 100 comprises a slider 700 that is extractable or retractable relative to the upper assembly 200 along the movement direction X (that is, along a movement axis X defined by the slider 700) through a plurality of equilibrium positions.

Looking in more detail, the slider 700 is extracted slidably in a direction that is the opposite of the patient's direction of movement while walking, and is retracted slidably in the same direction as the patient's direction of movement while walking.

The first end 600a of the connector 600 is pivoted to the slider 700—for example, hinged to it—to adjust the position of the first end 600a of the connector 600.

In other words, the position of the slider 700 along the movement direction X is adjustable (by extracting or retracting the slider 700) to position the first end 600a of the connector 600 at a predetermined distance from the upper assembly 200.

More specifically, the position of the first end 600a of the connector 600 can be adjusted between a most extracted position (FIG. 2C), where the first end 600a is distal to the upper assembly 200, and a least extracted position (FIG. 2A), where the first end 600a is proximal to the upper assembly 200. At the most extracted position, the prosthetic implant 100 exhibits a higher value of stiffness whilst, at the least extracted position, the prosthetic implant 100 exhibits a lower value of stiffness, as described in detail below. The first end 600a of the connector 600 is moved to the most extracted position when, for example, the patient lifts a load or walks on uneven ground and/or runs.

On the contrary, the first end 600a of the connector 600 is moved to the least extracted position when, for example, the patient needs to climb stairs or ramps or to walk on even ground.

Advantageously, the same prosthetic implant 100 is adaptable to the patient's different walking needs.

Also, the same prosthetic implant 100, being variable in stiffness, can be used by patients varying widely in body weight.

As shown in FIG. 2B, the slider 700 can also be moved in such a way that the first end 600a of the connector 600 occupies an intermediate position between the most extracted and the least extracted position. At such an intermediate position, the prosthetic implant 100 is in a standard walking configuration, that is to say, under conditions allowing the patient to walk in what may be termed everyday life conditions.

According to an aspect of this disclosure, the slider 700 is coupled to the upper assembly 200 by a prismatic joint. In this situation, the slider 700 is made in the form of a prismatic section bar slidably extractable from, or insertable into, a housing made on the upper assembly 200 and shaped to match the bar.

Also, the second end 600b of the connector 600 is articulated to the intermediate blade 400b of the lower assembly 300.

In the embodiment of FIGS. 1, 2A-2C and 3, the second end 600b of the connector 600 is articulated to the intermediate blade 400b by a hinge.

In use, therefore, for the prosthetic implant 100 to be able to simulate a human foot as faithfully as possible, that is to say, to replicate the force-deformation curve representing the patient's natural gait cycle, the slider 700 is extracted from the upper assembly 200 by a predetermined quantity. In this situation, the connector 600 varies its inclination relative to the longitudinal direction A.

For example, if the slider 700 is moved to the most extracted position, the connecting point (identified with the first end 600a of the connector 600) between the slider 700 and the connector 600 occupies a position distal to the upper assembly 200 and the connector 600 is substantially parallel to the longitudinal direction A. On the contrary, if the slider 700 is moved to the least extracted position, the connecting point (that is, the first end 600a of the connector 600) between the slider 700 and the connector 600 occupies a position close to the upper assembly 200 and the connector 600 is substantially inclined with respect to the longitudinal direction A.

According to an aspect of this disclosure, the change in position of the slider 700, hence of the first end 600a of the connector 600 along the movement direction X, is approximately 2 mm. In this situation, the stiffness of the prosthetic implant 100 changes by approximately 10 kg, allowing a good adjustment of its response to a change, for example, of the patient's weight. Since the blades 400a, 400b, 400c rest at least partly on each other and since they are laminated springs made from compositive material, their behaviour is a non-linear elastic behaviour. Changing the position of the first end 600a, that is, of the connecting point between the slider 700 and the connector 600, makes it possible to change the point of rotation of the blades 400a, 400b. 400c. In this situation, depending on the position of the first end 600a, the blades 400a, 400b, 400c are subjected to a different flexural state, thus allowing the elastic stiffness of the prosthetic implant 100 to be varied in non-linear manner.

Changing the position of the first end 600a of the connector 600 in a controlled manner varies the point where the force exchanged between the lower assembly 300 and the upper assembly 200 is applied. In this situation, the lever arm of the force relative to the centre of rotation of the ankle (that is, of the articulated joint 500) changes, hence the transmitted torque also changes.

In the embodiment of FIG. 1, the position of the first end 600a of the connector 600, hence the extent to which the slider 700 is extracted, can be adjusted manually (FIG. 1A).

In this situation, by using a adjustment tool, for example, an Allen key, it is possible to adjust the extent to which the slider 700 is extracted from the upper assembly 200, hence the position of the first end 600a of the connector 600 along the movement direction X.

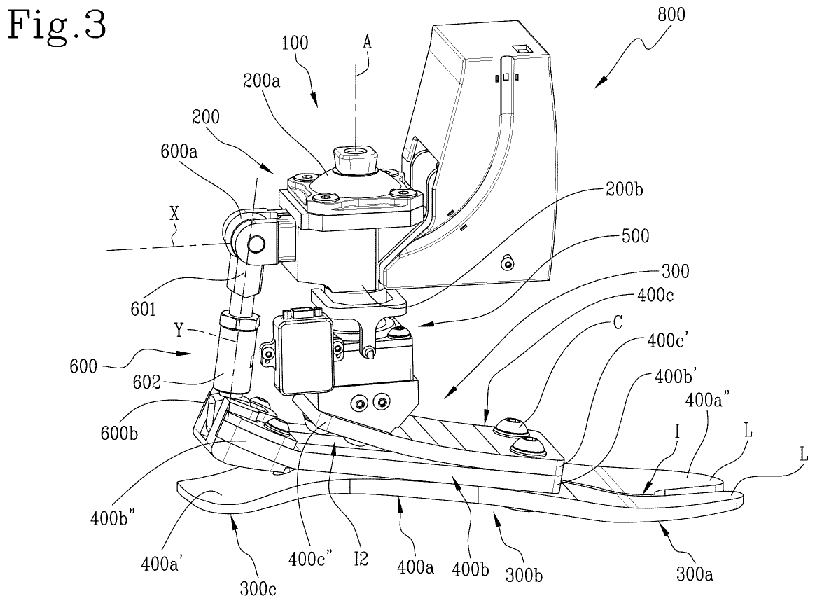

Alternatively, the prosthetic implant 100 comprises an actuator 800, shown in FIG. 3, configured to slidably move the first end 600a of the connector 600.

In another embodiment, shown in FIGS. 4 and 4A, the end that is moved is the second end 600b of the connector 600.

In this situation, the second end 600b of the connector 600 is slidably movable along the intermediate blade 400b, while the first end 600a is hinged or pivoted to the upper assembly 200.

Looking in more detail, as shown in FIG. 4A, the prosthetic implant 100 comprises a slide 901 that is slidably movable along the intermediate blade 400b to steplessly vary the position of the second end 600b of the connector 600 on the intermediate blade 400b.

The prosthetic implant 100 also comprises a locking element 902 that is applicable on the slide 901 to adjust the position of the slide 901 on the intermediate blade 400b.

According to an aspect of this disclosure, the locking element 902 can be applied to, and released from, the slide 901 to prevent or allow the slidable movement of the slide 901, respectively, so that its position along the intermediate blade 400b can be adjusted.

In the embodiment shown in the accompanying drawings, the locking element 902 is embodied in the form of a rotatable screw oriented substantially parallel to the intermediate blade 400b.

As shown in FIGS. 4 and 4A, the first end 600a of the connector 600 is pivoted to the upper assembly 200, while the second end 600b is engaged with the slide 901.

Adjusting the position of the second end 600b by sliding the slide 901 on the intermediate blade 400b makes it possible to vary the response of the prosthetic implant 100 so that it can adapt to different ground surface conditions in a manner almost identical to that of a natural foot.

In this situation, too, the force exchanged between the lower assembly 300 and the upper assembly 200 can be changed in a controlled manner by changing the position of the point where the force is applied, in this case the second end 600b of the connector 600. Changing the position of the point where the force is applied, changes the lever arm of the force relative to the centre of rotation of the ankle (that is, of the articulated joint 500), hence the transmitted torque. The variation in the torsional stiffness obtained can be attributed to the entire lower assembly 300.

Advantageously, the prosthetic implant 100 of this disclosure allows varying the stiffness of the lower assembly 300 non-linearly to adapt it to the patient's different walking needs.

More specifically, the lower assembly 300 constituting the foot is capable of faithfully replicating the force-deformation curve representing the behaviour of a natural foot when subjected to loads and/or when walking on uneven ground.

Another object of this disclosure is a method for adjusting a prosthetic implant 100 of the ESR (energy-storing-and-returning) type. The prosthetic implant 100 comprises an upper assembly 200 configured to be connected to a patient's lower limb, and a lower assembly 300 configured to define a foot having a front region 300a, a middle region 300b and a rear region 300c.

The lower assembly 300 includes a lower blade 400a extending along a main direction M, between a heel end 400a′ and a toe end 400a″; an intermediate blade 400b partly superposed on the lower blade 400a, and an upper blade 400c partly superposed on the intermediate blade 400b.

The blades 400a, 400b, 400c are laminated springs made from composite material.

The blades 400a, 400b, 400c have a non-linear elastic behaviour.

The upper, lower and intermediate blades 400a, 400b, 400c are fastened stably to each other at at least one connecting point C located in the front region 300a or in the middle region 300b of the foot and diverge from each other away from the connecting point C towards the rear region 300c of the foot.

More specifically, the upper blade 400c and the intermediate blade 400b are connected at the connecting point C at a toe end 400b′, 400c′, while the lower blade 400a is connected at the connecting point C in proximity to the middle region 300b of the foot.

The prosthetic implant 100 also comprises an articulated joint 500, interposed between the lower assembly 300 and the upper assembly 200 to allow relative motion between the lower assembly 300 and the upper assembly 200.

More specifically, the articulated joint 500 simulates an ankle articulation.

The prosthetic implant 100 also comprises a connector 600 having a first end 600a that is connected to the upper assembly 200 and a second end 600b that is connected to the intermediate blade 400b of the lower assembly 300 to apply a force on the intermediate blade 400b of the lower assembly 300.

The connector 600 is rigid and preferably comprises a first and a second portion connected to each other by a lead nut and screw connection.

The method comprises a step of moving at least one between the first and the second end 600a, 600b of the connector 600 to vary the force applied by the connector 600 on the intermediate blade 400b of the lower assembly 300.

Changing in a controlled manner the position of one between the first and the second end 600a, 600b of the connector 600, where the force is exchanged between the lower assembly 300 and the upper assembly 200 means changing the point where the force is applied, thereby changing the lever arm of the force relative to the centre of rotation of the ankle (that is, relative to the articulated joint 500), hence the torque transmitted.

Since the blades 400a, 400b, 400c interact (by resting on each other), their points of rotation vary with the changing of the load applied to them. Changing the position of one of the ends 600a, 600b of the connector 600 varies the lever arm of the force and the points of rotation, and thus also the rigidity of the prosthetic implant 100 is varied in non-linear elastic manner. In this situation, the prosthetic implant 100 can be adapted to the user's different needs such as, for example, walking on uneven ground, carrying weights and the like.

According to an aspect of this disclosure, the upper assembly 200 extends along a longitudinal direction A between a hook-up end 200a, where it is operatively connected to the patient's lower limb, and a fixing end 200b, where it is fixed to the articulated joint 500. In this situation, the method comprises a step of sliding the first end 600a of the connector 600 relative to the upper assembly 200. In this situation, the movement direction X is transverse to the longitudinal axis A.

Thanks to the step of sliding, the position of the first end 600a of the connector 600 can be adjusted relative to the upper assembly 200 in such a way as to vary the stiffness of the entire prosthetic implant 100.

According to an aspect of this disclosure, the prosthetic implant 100 comprises a slider 700 and the first end 600a of the connector 600 is pivoted to the slider 700.

In this situation, the method comprises a step of extracting or retracting the slider 700 relative to the upper assembly 200 along the movement direction X through a plurality of equilibrium positions, and a step of adjusting the position of the first end 600a of the connector 600 between a most extracted position, where the first end 600a is distal to the upper assembly 200, and a least extracted position, where the first end 600a is proximal to the upper assembly 200.

At the most extracted position, the stiffness of the prosthetic implant 100 is less than the stiffness when it is at the least extracted position.

Varying the position of the first end 600a of the connector 600by extracting or retracting the slider 700 varies the force exchanged between the lower assembly 300 and the upper assembly 200 via the slider 600. Varying the position of the first end 600a changes the point of application of the force, hence the lever arm of the force relative to the centre of rotation of the ankle (that is, relative to the articulated joint 500) is also varied, which in turn varies the torque transmitted.

In this situation, it is possible to adapt the foot to any type of ground and/or load which the patient needs to negotiate and/or support.

This invention achieves the preset aims and overcomes the disadvantages of the prior art.

In particular, the possibility of adjusting the position of one of the ends, specifically the first end, allows making the prosthetic implant adaptive, that is to say, it allows varying the stiffness of the prosthetic implant.

Moreover, the arrangement of the blades and their makeup allow varying the elastic stiffness of the prosthetic implant in non-linear manner, making the response of the prosthetic implant practically identical to the response of a foot of an able-bodied patient walking on different types of ground.

1. A prosthetic implant of the ESR (energy-storing-and-return) type, comprising:
an upper assembly configured to be connected to a patient's lower limb;
a lower assembly, configured to define a foot having a front region, a middle region and a rear region and including:
a lower blade, extending along a main direction, between a heel end and a toe end;
an intermediate blade, partly superposed on the lower blade;
an upper blade, partly superposed on the intermediate blade;
wherein the upper, lower and intermediate blades are fastened stably to each other at at least one connecting point located in the front region or in the middle region of the foot and diverge from each other away from the connecting point towards the rear region of the foot;
an articulated joint, interposed between the lower assembly and the upper one to allow relative motion between the lower assembly and the upper one;
a connector having a first end that is connected to the upper assembly and a second end that is connected to the intermediate blade of the lower assembly to apply a force on the intermediate blade of the lower assembly,
wherein at least one between the first and the second end of the connector is movable along a movement direction to vary the force applied on the intermediate blade of the lower assembly.

2. The implant according to claim 1, wherein the upper assembly extends along a longitudinal direction between a hook-up end where it is operatively connected to the patient's lower limb, and a fixing end, where it is fixed to the articulated joint, and wherein the first end of the connector is slidably movable relative to the upper assembly, the movement direction being transverse to the longitudinal axis.

3. The implant according to claim 2, comprising a slider, extractable or retractable relative to the upper assembly along the movement direction through a plurality of equilibrium positions, the first end of the connector being pivoted to the slider to adjust the position of the first end of the connector between a most extracted position, where the first end is distal to the upper assembly, and a least extracted position, where the first end is proximal to the upper assembly.

4. The implant according to claim 3, wherein the slider is coupled to the upper assembly by a prismatic joint.

5. The implant according to claim 1, wherein the second end of the connector is articulated to the intermediate blade of the lower assembly.

6. The implant according to claim 1, comprising an actuator configured to slidably move the first end of the connector.

7. The implant according to claim 1, wherein the second end of the connector is slidably movable along the intermediate blade.

8. The implant according to claim 7, comprising:
a slide that is slidably movable along the intermediate blade to steplessly vary the position of the second end of the connector on the intermediate blade;
an adjustment element that is activable on the slide to adjust the position of the slide on the intermediate blade.

9. The implant according to claim 1, wherein the connector is a rigid element and wherein the length of the connector is adjustable.

10. The implant according to claim 1, wherein the articulated joint includes:
a ball that is stably fixed to the upper assembly and faces towards the lower assembly;
a receiving body that is fixed to the lower assembly and faces towards the upper assembly to receive the ball;
the ball and the receiving body defining one between a ball and socket joint, a rotoidal joint or a universal joint.

11. The implant according to claim 1, wherein the lower blade, the intermediate blade and the upper blade are laminated springs made from composite material.

12. The implant according to claim 1, wherein
the connector extends from the first end to the second end along a connection axis;
the movement direction is inclined with respect to the connection axis.

13. The implant according to claim 1, wherein
the upper assembly extends along a longitudinal direction between a hook-up end, where it is operatively connected to the patient's lower limb, and a fixing end, where it is fixed to the articulated joint;
the movement direction is perpendicular to the longitudinal direction.

14. A method for adjusting a prosthetic implant of the ESR (energy-storing-and-return) type, the prosthetic implant comprising
an upper assembly configured to be connected to a patient's lower limb;
a lower assembly, configured to define a foot having a front region, a middle region and a rear region, wherein the lower assembly includes:
a lower blade, extending along a main direction, between a heel end and a toe end;
an intermediate blade, partly superposed on the lower blade;
an upper blade, partly superposed on the intermediate blade;
wherein the upper, lower and intermediate blades are fastened stably to each other at at least one connecting point located in the front region or in the middle region of the foot and diverge from each other away from the connecting point towards the rear region of the foot;
an articulated joint, interposed between the lower assembly and the upper assembly to allow relative motion between the lower assembly and the upper assembly;
a connector having a first end that is connected to the upper assembly and a second end that is connected to the intermediate blade of the lower assembly to apply a force on the intermediate blade of the lower assembly,
wherein the method comprises a step of moving at least one between the first and the second end of the connector along a movement direction to vary the force applied by the connector on the intermediate blade of the lower assembly.

15. The method according to claim 14, wherein the upper assembly extends along a longitudinal direction between a hook-up end, where it is operatively connected to the patient's lower limb, and a fixing end, where it is fixed to the articulated joint, the method comprising a step of sliding the first end of the connector relative to the upper assembly, the movement direction being transverse to the longitudinal axis.

16. The method according to claim 15, where the implant comprises a slider and wherein the first end of the connector is pivoted to the slider, and wherein the method comprises the following steps:
extracting or retracting the slider relative to the upper assembly along the movement direction through a plurality of equilibrium positions;
adjusting the position of the first end of the connector between a most extracted position, where the first end is distal to the upper assembly, and a least extracted position, where the first end is proximal to the upper assembly.

17. The method according to claim 14, wherein
the connector extends from the first end to the second end along a connection axis;
the movement direction is inclined with respect to the connection axis.

18. The method according to claim 14, wherein
the upper assembly extends along a longitudinal direction between a hook-up end, where it is operatively connected to the patient's lower limb, and a fixing end, where it is fixed to the articulated joint;
the movement direction is perpendicular to the longitudinal direction.

19. A prosthetic implant of the ESR, energy-storing-and-returning, type, comprising:
an upper assembly for connection to a patient's lower limb;
a lower assembly, defining a foot having a front region, a middle region and a rear region and including:
a lower blade, extending along a main direction, between a heel end and a toe end;
an intermediate blade, partly superposed on the lower blade;
an upper blade, partly superposed on the intermediate blade;
wherein the upper, lower and intermediate blades are fastened stably to each other at a connecting point and diverge from each other away from the connecting point towards the rear region of the foot;
an articulated joint, interposed between the lower assembly and the upper assembly to allow relative motion between the lower assembly and the upper assembly;
a connector extending from a first end to a second end along a connection axis, the first end being connected to the upper assembly and the second end being connected to the intermediate blade of the lower assembly to apply a force thereon,
wherein at least one between the first end and the second end of the connector is movable along a movement direction, the movement direction being inclined with respect to the connection axis, so to adjust the force applied on the intermediate blade of the lower assembly.

20. The implant according to claim 19, wherein
the upper assembly extends along a longitudinal direction between a hook-up end, operatively connected to the patient's lower limb, and a fixing end, the upper assembly being fixed to the articulated joint at the fixing end;
the movement direction is perpendicular to the longitudinal direction.
